(19) United States
(12) Patent Application Publication
(10) Pub. No.: US 2013/0040397 A1
(43) Pub. Date: Feb. 14, 2013

(54) DETECTION OF HYDROGEN SULFIDE GAS USING CARBON NANOTUBE-BASED CHEMICAL SENSORS

(71) Applicants: ALEXANDER STAR, Pittsburgh, PA (US); Mengning Ding, Pittsburgh, PA (US)

(72) Inventors: ALEXANDER STAR, Pittsburgh, PA (US); Mengning Ding, Pittsburgh, PA (US)

(21) Appl. No.: 13/251,811

(22) Filed: Oct. 3, 2011

Related U.S. Application Data
(60) Provisional application No. 61/388,843, filed on Oct. 1, 2010.

(51) Int. Cl. G01N 27/04; G01N 31/00; B32B 9/04; C07C 303/20; B05D 5/12; B82Y 5/00; B82Y 15/00; B82Y 40/00

(52) U.S. Cl. 436/121; 422/98; 562/90; 436/149; 427/122; 428/408; 977/762; 977/742; 977/896; 977/788; 977/904

(57) ABSTRACT

A method for preparing carbon allotrope based sulfide detectors comprising first functionalizing a carbon allotrope, such as a single-walled carbon nanotubes or graphene, with a solution of a polynuclear aromatic hydrocarbon-sulfonic acid, such as 1-pyrenesulfonic acid, followed by treatment with a metal, such as gold nanowires or cupric salt doped polyaniline, to give a metal-functionalized carbon allotrope, then drop casting the metal-functionalized carbon allotrope onto an inert surface, such as a silicon dioxide film on a silicon wafer having electrodes. Detection of sulfides may be by means such as photochemical or conductance methods. The hydrogen sulfide detectors may be used to detect and/or quantitate ppb and ppm levels of hydrogen sulfide in industrial settings or in detecting halitosis.

### FIELD OF INVENTION

This invention describes detection of hydrogen sulfide gas using carbon nanotube-based chemical sensors.

### BACKGROUND OF THE INVENTION

Hydrogen sulfide is a corrosive, toxic, inflammable and odoriferous chemical that causes safety concerns. The threshold limit value (THL) and the recommended exposure limit (REL) of hydrogen sulfide are both set at 10 ppm, and it is life-threatening when exceeding 300 ppm. In addition to public safety, hydrogen sulfide can affect personal and social communications. Presence of this chemical in breath at concentrations of 300 ppb and higher is responsible for halitosis (bad breath).

Detection of hydrogen sulfide is therefore important for applications in industrial monitoring, personal safety, and medical field. Commercially available hydrogen sulfide detectors are often based on electrochemical methods or specific spectroscopic techniques, while solid-state resistivity-based sensors can offer certain advantages for development of portable and low-cost hydrogen sulfide detectors.

In the current market, most portable hydrogen sulfide detectors are based on electrochemical sensing (which requires multiple electrodes and electrolyte solution) and usually have a detection limit of no less than 1 ppm (not sufficient for some applications such as monitoring of human breath odor). Other hydrogen sulfide detectors that can reach the parts-per-billion (ppb) concentration levels are based on spectroscopic techniques such as cavity ring down spectroscopy, CDRS, which require complicated instrumentation and sophisticated equipment.

Solid-state gas sensors comprising carbon nanotubes, especially single-walled carbon nanotubes (SWNTs), offer a unique technical solution for the development of solid-state gas sensors, due to advanced properties of SWNTs such as high surface area, physical robustness, chemical inertness and electrical conductivity that is sensitive to perturbations in their local chemical environment. However, bare SWNTs show little or no response to hydrogen sulfide.

One-dimensional conducting nanomaterials have unique properties such as tunable electrical conductivity and high surface area, which make them attractive for chemical sensing. For example, carbon nanotubes, and especially SWNTs, which are composed entirely of surface atoms, possess non-selective electric properties that are extremely sensitive to perturbations in their local environment. Another one-dimensional nanomaterial, polyaniline nanofibers (PAni-nanofibers), demonstrates sensitivity towards many analytes due to possible transformations between different forms of PAni. Additionally, the PAni-nanofiber structure enables sensor films with high porosity that results in excellent sensitivity and response time. However, PAni-nanofibers have limited chemical stability, which is a common problem of all organic conductors. It is, therefore, of great interest to explore whether the combination of these two one-dimensional nanomaterials provide any additional advantages for the development of novel chemical sensors.

### SUMMARY OF THE INVENTION

Bare SWNTs show little or no response to hydrogen sulfide, thus chemical functionalization is needed in order to induce significant hydrogen sulfide sensitivity. Sulfide sensors based on the functionalized carbon allotropes, such as graphene and single-walled carbon nanotubes as in this invention, are an effective solution to combine the features of high sensitivity and portability.

A method is provided for preparing a sulfide sensitive carbon allotrope. The method comprises the steps of first functionalizing a carbon allotrope having sp² bonded carbon atoms with a solution of a polynuclear aromatic hydrocarbon-sulfonic acid, wherein an aqueous suspension of the first-functionalized carbon allotrope are formed, and second functionalizing the first-functionalized carbon allotrope in the aqueous solution with a metal, whereby a sulfide sensitive carbon allotrope is formed.

A method is provided for preparing a sulfide chemical sensor. The method comprises the steps of first functionalizing a carbon allotrope having sp² bonded carbon atoms with a solution of a polynuclear aromatic hydrocarbon-sulfonic acid, wherein an aqueous suspension of the first-functionalized carbon allotrope are formed, and second functionalizing the first-functionalized carbon allotrope in the aqueous solution with a metal to give the sulfide sensitive carbon allotrope. Further, the sulfide sensitive carbon allotrope may be deposited onto an inert surface.

A composite is provided comprising a carbon allotrope having sp² bonded carbon atoms; on a surface of the carbon allotrope is a polynuclear sulfonic acid, and on the polynuclear sulfonic acid is a sulfide reactive material.

A chemical sensor is provided comprising a composite comprised of a carbon allotrope having sp² bonded carbon atoms, the carbon allotrope disposed on an inert surface; a polynuclear sulfonic acid disposed on the carbon allotrope, and disposed on the polynuclear sulfonic acid is a sulfide reactive material.

A method of use of a chemical sensor is provided comprising exposing a composite comprised of sulfide reactive material disposed on a polynuclear sulfonic acid, the polynuclear sulfonic acid disposed on a carbon allotrope having sp² bonded carbon atoms, and the carbon allotrope disposed on an inert material, to a sulfide compound; and then measuring the response of the sensor to the sulfide compound.

In the above methods, composites, and sensors, the polynuclear aromatic hydrocarbon-sulfonic acid comprises at least two aromatic rings. Preferably, the polynuclear aromatic hydrocarbon comprises naphthalene, anthracene, phenanthrene, pyrene, or benzopyrene. And more preferably, the polynuclear aromatic hydrocarbon comprises pyrene, and yet more preferably, the polynuclear aromatic hydrocarbon-sulfonic acid is 1-pyrene sulfonic acid.

In the above methods, composites, and sensors, the metal preferably comprises a noble metal and wherein the second functionalization comprises a deposition of the noble metal and nanowelding the noble metal into noble metal nanowire from the metal. The noble metal may be ruthenium, rhodium, palladium, silver, osmium, iridium, platinum, or gold. The noble metal may be a combination of one or more noble metals. Preferably the noble metal is platinum or gold. More preferably the noble metal is platinum. Even more preferably, the noble metal comprises gold. Nanowelding may be carried out by heating.

In the above methods, composites, and sensors, the metal comprises a cupric salt and wherein the second functionalization comprises a polymerization of aniline onto a surface of the polynuclear aromatic hydrocarbon-sulfonic acid. The cupric salt may be doped into the polyaniline. Preferably, the cupric salt is cupric chloride.

In the above methods, composites, and sensors, the carbon allotrope may be planar sheets or cylindrical tubes of sp² bonded carbon atoms. Preferably the carbon allotrope is one or more two-dimensional sheets of graphite as a graphene. More preferably, the carbon allotrope has a cylindrical nanostructure. Even more preferably, the carbon allotrope is a single-walled carbon nanotube as a graphene.

In the above methods, composites, and sensors, the inert surface may be silicon dioxide. The inert surface may be supported by a base surface. The base surface may comprise silicon. The inert surface may comprise at least one electrode. Preferably, the inert surface comprises a plurality of electrodes.

In the above composites, methods, and sensors, the materials are sensitive to the presence of sulfides. The sulfide compound may be a dialkyl sulfide, a diaryl sulfide, an alkyl aryl sulfide, an alkyl hydrogen sulfide, an aryl hydrogen sulfide, or dihydrogen sulfide (hydrogen sulfide). Preferably the sulfide comprises hydrogen sulfide. The sulfide compound may be comprised of a combination of one or more different sulfide compounds.

### DRAWINGS

FIG. 1 is a schematic representation of a composite of one embodiment of the present invention;

FIG. 2 is a schematic representation of a chemical sensor of one embodiment of the present invention;

FIG. 3 shows a chemical sensor according to a preferred embodiment of the present invention and associated analytical data of the chemical sensor;

FIG. 4 demonstrates of analytical response of a preferred embodiment of the present invention to pulses of humidity;

FIG. 5 is a representation of a control experiment wherein metal nanoparticles deposited without the presence of a carbon allotrope;

FIG. 6 is a schematic representation self-assembly and nanowelding of a preferred method of the present invention;

FIG. 7 comprises a comparison of two transmission electron microscope (TEM) photographs, the first a nanowelding of a preferred embodiment of the present invention and the second a control experiment wherein first functionalization utilized 1-pyrene carboxylic acid;

FIG. 8 is a schematic representation of a method of preparation and use of a preferred chemical sensor of the present invention, in addition to a TEM of nanowires and analytical data of the chemical sensor of the present invention;

FIG. 9 is a schematic representation of a method of preparation and use of another embodiment of a composite of the present invention and associated analytical data of the embodiment;

FIG. 10 is an analytical comparison of a preferred embodiment of a chemical sensor of the present invention compared to a control; and

FIG. 11 is a schematic representation of a preferred embodiment of a chemical sensor of the present invention demonstrating robustness.

### DETAILED DESCRIPTION OF PREFERRED EMBODIMENTS OF THE INVENTION

In the following detailed description, reference is made to the accompanying examples and figures that form a part hereof, and in which is shown, by way of illustration, specific embodiments in which the inventive subject matter may be practiced. These embodiments are described in sufficient detail to enable those skilled in the art to practice them, and it is to be understood that other embodiments may be utilized and that structural or logical changes may be made without departing from the scope of the inventive subject matter. Such embodiments of the inventive subject matter may be referred to, individually and/or collectively, herein by the term “invention” merely for convenience and without intending to voluntarily limit the scope of this application to any single invention or inventive concept if more than one is in fact disclosed. The following description is, therefore, not to be taken in a limited sense, and the scope of the inventive subject matter is defined by the appended claims and their equivalents.

As set forth herein are a number of preferred strategies to functionalize SWNTs with different nanomaterials and nanostructures for the development of portable solid state hydrogen sulfide detectors.

The first aspect of the invention is a method of formation of a sulfide sensitive carbon allotrope. The method comprises the steps of layering a polynuclear sulfonic acid on a surface of a carbon allotrope having pi orbitals whereby the polynuclear sulfonic acid may interact by pi-pi stacking with the carbon allotrope; and functionalizing an exposed surface of the polynuclear sulfonic acid with a sulfide reactive material.

A first embodiment of the first aspect of the invention comprises the formation of a noble metal nanowire as the sulfide reactive material. The method comprises the steps of layering a polynuclear sulfonic acid on a surface of a carbon allotrope having pi orbitals whereby the polynuclear sulfonic acid may interact by pi-pi stacking with the carbon allotrope; reductively depositing a noble metal from a solution onto the polynuclear sulfonic acid to form nanoparticles; and nanowelding the nanoparticles into nanowires.

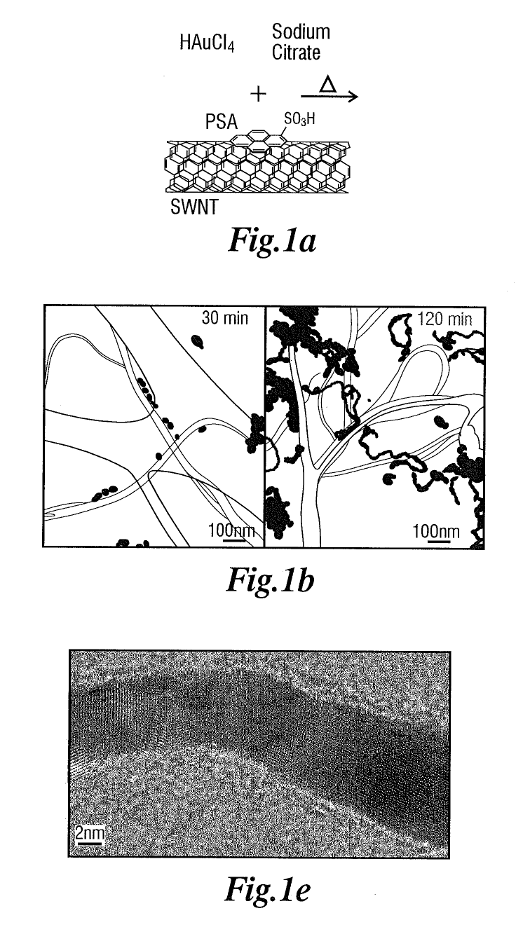

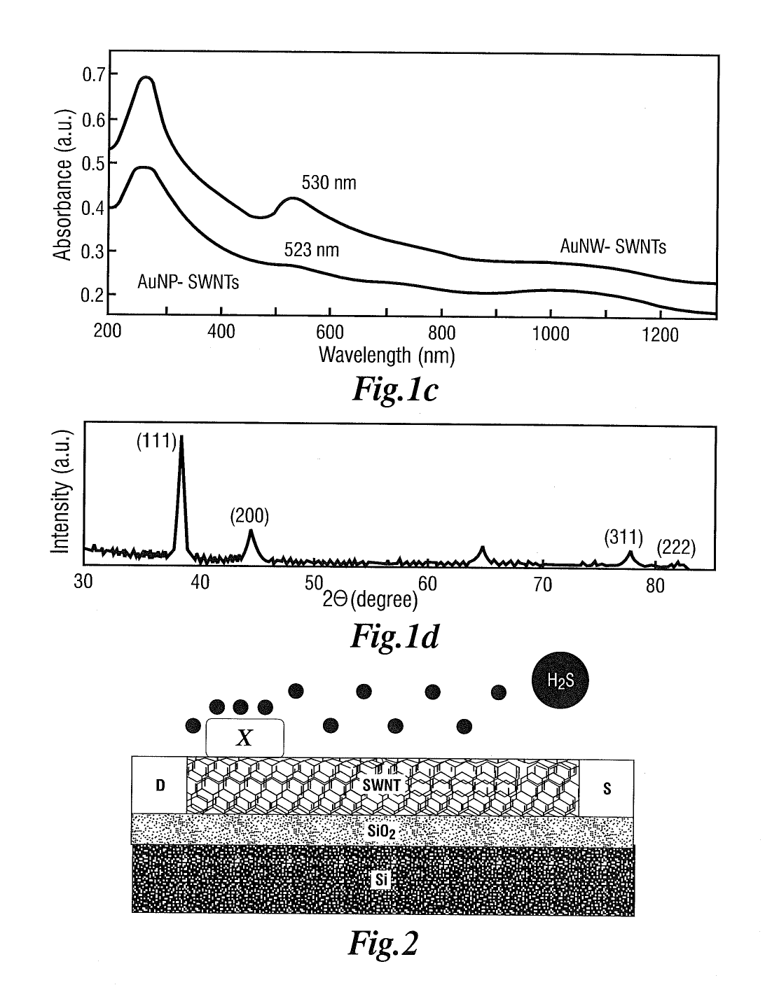

Reductive deposition of a noble metal of an aqueous solution may be used to deposit the noble metal as individual particles onto the surface of the carbon allotrope as shown in FIG. 1a. The nanoparticles may be seen as discrete particles after 30 minutes in FIG. 1b (30 min). Nanowelding may be carried out with heating. Nanowelding forms nanowires of a noble metal, for example by heating for 120 minutes, as shown in FIG. 1b (120 min).

The formation of the nanowires by the method of the first aspect may be accompanied by a spectral shift, as shown in FIG. 1c. The nanowires prepared by the first aspect may exhibit a polycrystalline nature as seen in the x-ray diffraction pattern of FIG. 1d and the high resolution TEM image of a gold nanowire shown in FIG. 1e.

The carbon allotrope may be planar sheets or cylindrical tubes of sp² bonded carbon atoms. Preferably the carbon allotrope is one or more two-dimensional sheets of graphite as a graphene. More preferably, the carbon allotrope is a single-walled carbon nanotube as a graphene. Even more preferably, the carbon allotrope has a cylindrical nanostructure.

The polynuclear sulfonic acid may have two or more fused aromatic rings and at least one sulfonic acid functional group. Preferably, the polynuclear sulfonic acid has three or four fused aromatic rings. Still other polynuclear sulfonic acids have no more than two sulfonic acid functional groups. More preferably, the polynuclear sulfonic acid is a pyrenesulfonic acid. Even more preferably, the polynuclear sulfonic acid is 1-pyrenesulfonic acid. The polynuclear sulfonic acid may be reacted with the carbon allotrope in an aqueous suspension.

The noble metal may be ruthenium, rhodium, palladium, silver, osmium, iridium, platinum, or gold. The noble metal may be a combination of one or more noble metals. Preferably the noble metal is platinum or gold. More preferably the noble metal is platinum. Even more preferably, the noble metal is gold.

The reductive deposition of the noble metal may be carried out by any known means. Known means are not limited to chemical vapor deposition, electrochemical deposition or chemical reductive deposition. Preferably the reductive deposition is by chemical reductive deposition means. The reductive deposition may be carried out with an aqueous solution of a borohydride. More preferably, the reductive deposition may be an aqueous solution of sodium borohydride and chloroplatinic acid. The reductive deposition may be a reaction of an aqueous solution of a noble metal salt with citric acid. Even more preferably, the reductive deposition is a reaction of an aqueous solution of a chloroauric acid and sodium citrate. Still more preferably, the reductive deposition is carried out with the addition of heat. Addition of heat increases the rate of formation of nanowires from the noble metal nanoparticles. Preferably, the heating is at a temperature of about 100° C.

A second embodiment of the first aspect of the invention comprises the formation of a cupric salt doped polyaniline as the sulfide reactive material. The method comprises the steps of layering a polynuclear sulfonic acid on a surface of a carbon allotrope having pi orbitals whereby the pi orbitals of a polynuclear sulfonic acid may interact by pi-pi stacking with the pi orbitals of the carbon allotrope; polymerizing aniline onto the polynuclear sulfonic acid; and incorporating cupric chloride into the polyaniline layer.

The carbon allotrope may be planar sheets or cylindrical tubes of sp² bonded carbon atoms. Preferably the carbon allotrope is one or more two-dimensional sheets of graphite as a graphene. More preferably, the carbon allotrope has a cylindrical nanostructure. Even more preferably, the carbon allotrope is a single-walled carbon nanotube as a graphene.

The polynuclear sulfonic acid may have two or more fused aromatic rings and at least one sulfonic acid functional group. Preferably, the polynuclear sulfonic acid has three or four fused aromatic rings. Still other polynuclear sulfonic acids have no more than two sulfonic acid functional groups. More preferably, the polynuclear sulfonic acid is a pyrenesulfonic acid. Even more preferably, the polynuclear sulfonic acid is 1-pyrenesulfonic acid.

As illustrated in FIG. 2, a second aspect of the present invention is a method of formation of a sulfide sensor. The method comprises the steps of depositing a polynuclear sulfonic acid on a surface of a carbon allotrope having pi orbitals whereby the polynuclear sulfonic acid may interact by pi-pi stacking with the carbon allotrope; functionalizing an exposed surface of the polynuclear sulfonic acid with a sulfide reactive material to form a sulfide sensitive carbon allotrope; depositing the sulfide sensitive carbon allotrope onto an inert surface. Preferably, the functionalizing of the exposed surface comprises the first or second embodiment of the first aspect.

Preferably the inert surface comprises silicon dioxide. Even more preferably, the inert surface is layered with the base surface comprising silicon, on the silicon is a layer comprising silicon dioxide, and on the opposite surface of the silicon dioxide is the deposited carbon allotrope. More preferably the inert surface contains one or more electrodes, see electrodes D and S in FIG. 2.

A third aspect of the invention is a composition comprising a carbon allotrope, on a surface of the carbon allotrope is a polynuclear sulfonic acid, and on an exposed surface of the polynuclear sulfonic acid is a sulfide reactive material. In a first embodiment of the third aspect, the sulfide reactive material is a noble metal nanowire. In a second embodiment of the third aspect, the sulfide reactive material is a polyaniline doped with a cupric salt.

The carbon allotrope may be planar sheets or cylindrical tubes of sp² bonded carbon atoms. Preferably the carbon allotrope comprises one or more two-dimensional sheets of graphite as a graphene. More preferably, the carbon allotrope comprises a cylindrical nanostructure. Even more preferably, the carbon allotrope comprises a single-walled carbon nanotube as a graphene.

The polynuclear sulfonic acid may have two or more fused aromatic rings and at least one sulfonic acid functional group. Preferably, the polynuclear sulfonic acid has three or four fused aromatic rings. Still other polynuclear sulfonic acids comprise no more than two sulfonic acid functional groups. More preferably, the polynuclear sulfonic acid comprises a pyrenesulfonic acid. Even more preferably, the polynuclear sulfonic acid comprises 1-pyrenesulfonic acid.

The noble metal may be ruthenium, rhodium, palladium, silver, osmium, iridium, platinum, or gold. The noble metal may comprise a combination of one or more noble metals. Preferably the noble metal comprises platinum or gold. More preferably the noble metal comprises platinum. Even more preferably, the noble metal comprises gold.

The fourth aspect of the present invention is a chemical sensor. The chemical sensor comprises the composition of the third aspect deposited on an inert surface. Preferably, the inert surface comprises silicon dioxide. More preferably, at least one electrode is present in the inert surface. Even more preferably, the inert surface is sandwiched between the composition of the third aspect and a silicon wafer.

A fifth aspect of the present invention is the method of use of the sulfide sensor of the fourth aspect. The method comprises exposing a sensor a carbon allotrope comprising sp² bonded carbon atoms, the carbon allotrope disposed on an inert surface, a polynuclear sulfonic acid disposed on the carbon allotrope, and a sulfide reactive material substantially disposed on the polynuclear sulfonic acid; and determining if there has been a response of the sensor to the sulfide compound. The response may comprise a spectrophotometrical change. More preferably, the response may comprise a change to conductance or resistance between the electrodes. Even more preferably, the spectrophotometrical or electrical change is measured before and after exposure of the chemical sensor to the sulfide compound.

In any of the aspects of the invention, the sulfide compound may be comprised of a dialkyl sulfide, a diaryl sulfide, an alkyl aryl sulfide, an alkyl hydrogen sulfide, an aryl hydrogen sulfide, or dihydrogen sulfide (hydrogen sulfide). The sulfide may contain other functional groups. Preferably, the sulfide compound comprises an aryl hydrogen sulfide, more preferably the sulfide compound comprises thiophenol. Still more preferably, the sulfide compound comprises a dialkyl sulfide, preferably, the dialkyl sulfide comprises dimethylsulfide. Even more preferably, the sulfide compound comprises an alkyl hydrogen sulfide, preferably, the alkyl hydrogen sulfide comprises methanethiol. Yet even more preferably the sulfide comprises dihydrogen sulfide. The sulfide compound may be comprised of a combination of one or more different sulfide compounds.

FIG. 2 illustrates a schematic representation of electrical detection of hydrogen sulfide using SWNTs in a preferred embodiment of the present invention. SWNTs were functionalized with different functionalization materials “X”, which have specific affinities to hydrogen sulfide molecules. Upon exposure to hydrogen sulfide, interaction between “X” and hydrogen sulfide will cause a significant change in the electrical conductivity of SWNT and enable hydrogen sulfide detection. In preferred embodiments, chemical functionalization of SWNT was carried out in solution phase, and the functionalized SWNT were then drop-casted onto a silicon dioxide film grown on a silicon wafer with inter-digitated gold electrodes. For gas detection, electrical conductivity (or resistivity) of functionalized SWNT may be measured using a programmable digital source-meter under a direct voltage supply. Upon exposure to hydrogen sulfide, the conductivity of the functionalized SWNT changes due to the interaction between hydrogen sulfide molecules and the functionalized SWNT. This change in device conductivity is proportional to hydrogen sulfide concentration.

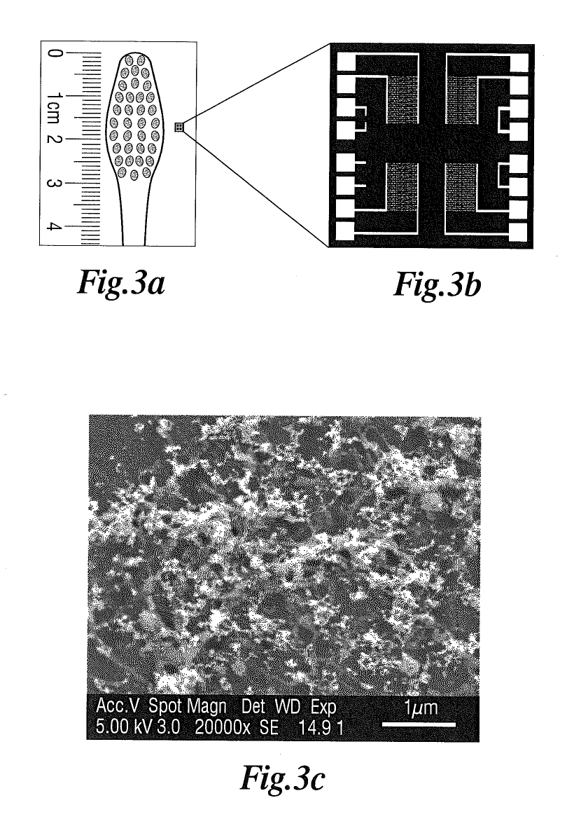

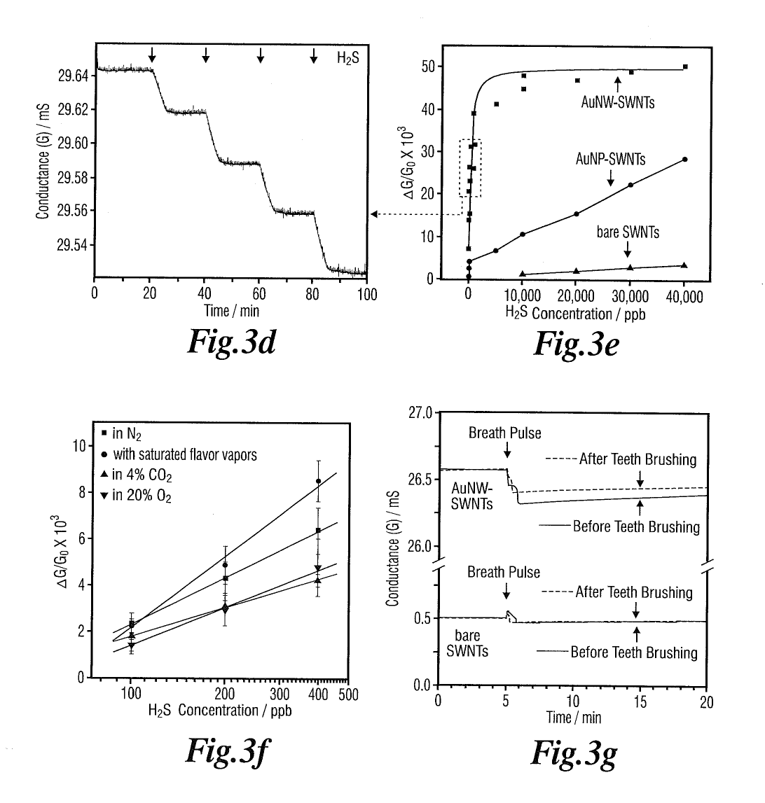

Hydrogen Sulfide (H₂S) Detection. A gold-nanowire-SWNT sensor device of a preferred embodiment of the present invention was fabricated by drop-casting the gold-nanowire-SWNT suspension onto a Si chip with interdigitated gold electrodes; the size, pattern and SEM images of a typical device was shown in FIG. 3a-c. The hydrogen sulfide response of gold-nanowire-SWNTs was tested, and the data (FIG. 3d) indicated that conductance of the gold-nanowire-SWNT devices significantly decreased when exposed to hydrogen sulfide at the concentration range from 10 ppb to 40 ppm (diluted in N₂). In a nitrogen environment at room temperature, the detection limit appeared to be lower than 10 ppb, which meets the sensitivity requirement for a variety of applications ranging from mine safety to the detection of breath odor. The recommended exposure limit (REL) to hydrogen sulfide is 10 ppm, and the presence of hydrogen sulfide at 500 ppb (or higher) in human breath will cause halitosis (breath odor). The cross-sensitivity of gold-nanowire-SWNTs to other major components of human breath (O₂, CO₂, water, ethanol and artificial flavor compounds) were also tested, and no obvious cross sensitivity was observed (FIG. 3f).

As a comparison, gold-nanoparticle-SWNT devices fabricated by electrochemical deposition were tested for response to hydrogen sulfide at both the ppb and ppm levels (FIG. 3e). At the tested hydrogen sulfide concentration ranges, gold-nanowire-SWNTs appeared to have better sensitivity to hydrogen sulfide, especially in the ppb level, with a detection limit for hydrogen sulfide of about one order of magnitude lower than that of gold-nanoparticle-SWNT. As a comparison, hydrogen sulfide tests were also conducted on pristine SWNTs (FIG. 3e).

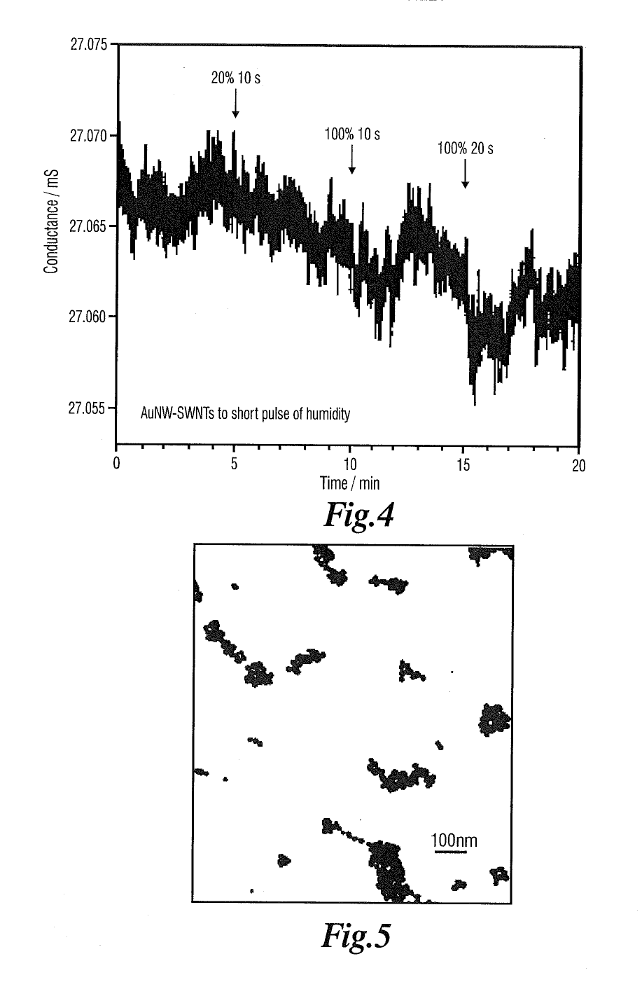

Human Breath (Odor) Detection. The real-time electrical response of gold-nanowire-SWNTs of a preferred embodiment of the present invention to human breath samples was tested. No special treatment was necessary for the breath samples, and signals were obtained simply by breathing towards the gold-nanowire-SWNT sensor device operated in an ambient environment. As shown in FIG. 3g, gold-nanowire-SWNTs demonstrated a significant decrease in the conductance when a person breathed towards the device thus indicating that the device was capable of detecting bad breath. Moreover, the sensor response was different before and after teeth brushing thereby showing a potential application for gold-nanowire-SWNTs in the detection of bad breath for the self-diagnosis of halitosis. Other control tests were completed to rule out false signals including short pulses (10 seconds) of high humidity vapors, where no significant response was observed for the gold-nanowire-SWNTs (FIG. 4). As a control, bare SWNTs were also employed to test bad breath; however, only a pressure response was observed (FIG. 3g).

As another control experiment, aqueous chloroauric acid reduced without the presence of SWNTs in the system, and a representative TEM image of the product is shown in FIG. 5. Without SWNTs in the reaction mixture, the gold nanowires were not observed under similar synthetic conditions; only aggregates of gold nanoparticles were present (with some of them already fused together). This indicates the necessity of utilizing the carbon allotrope as a template for the formation of nanowire structures.

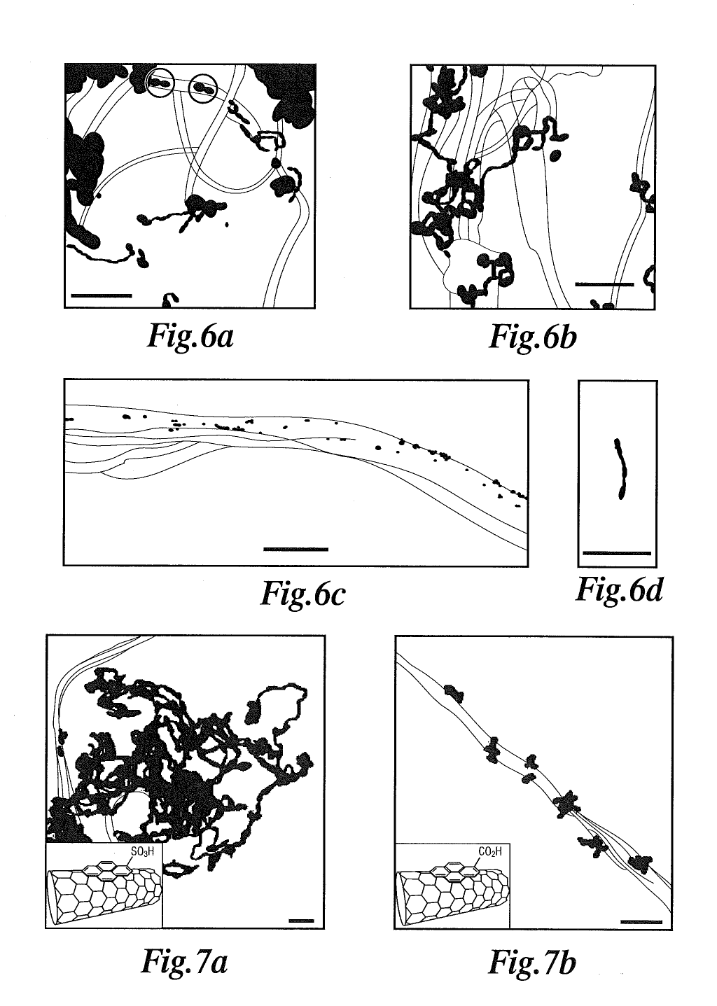

In still another control experiment, commercially available gold colloidal solution (Sigma Aldrich) was used as the pre-fabricated building blocks with a SWNT template. Both self-assembly of the nanoparticles and the nanowelding process were observed as in FIG. 6a-c, demonstrating that self-assembly and nanowelding does not require the in situ synthesis of the gold nanoparticles in the system.

In yet another control experiment, the method of the second aspect was carried out substituting 1-pyrene carboxylic acid for the 1-pyrenesulfonic acid. Small aggregates of several nanoparticles were observed after the welding process (FIG. 7a) instead of nanowires (FIG. 7b).

### Example 1

### “X”=Gold Nanowires (AuNW)

Gold nanowire functionalized SWNTs of a preferred embodiment of the present invention were synthesized by first functionalizing single-walled carbon nanotubes with 1-pyrenesulfonic acid (PSA) to produce a uniform aqueous suspension, and then citrate reduction of chloroauric acid in the single-walled carbon nanotube suspension. In this preferred embodiment, the citrate reduction was done in situ.

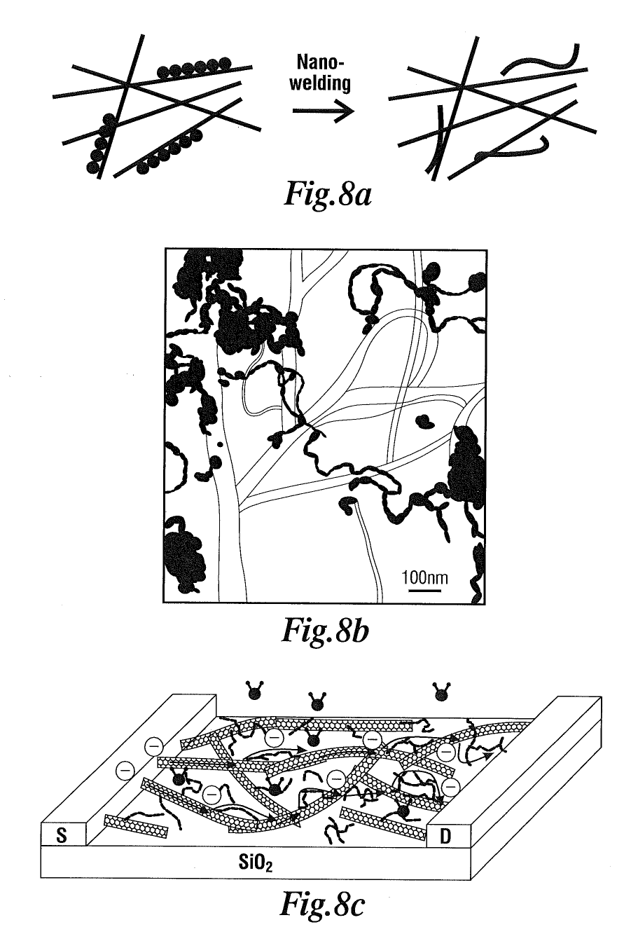

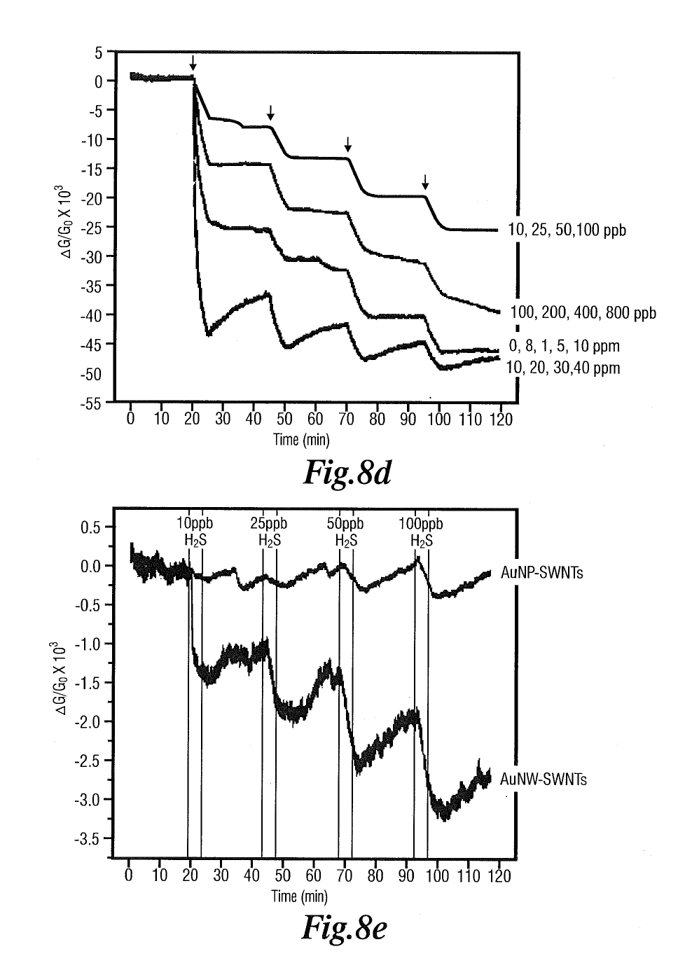

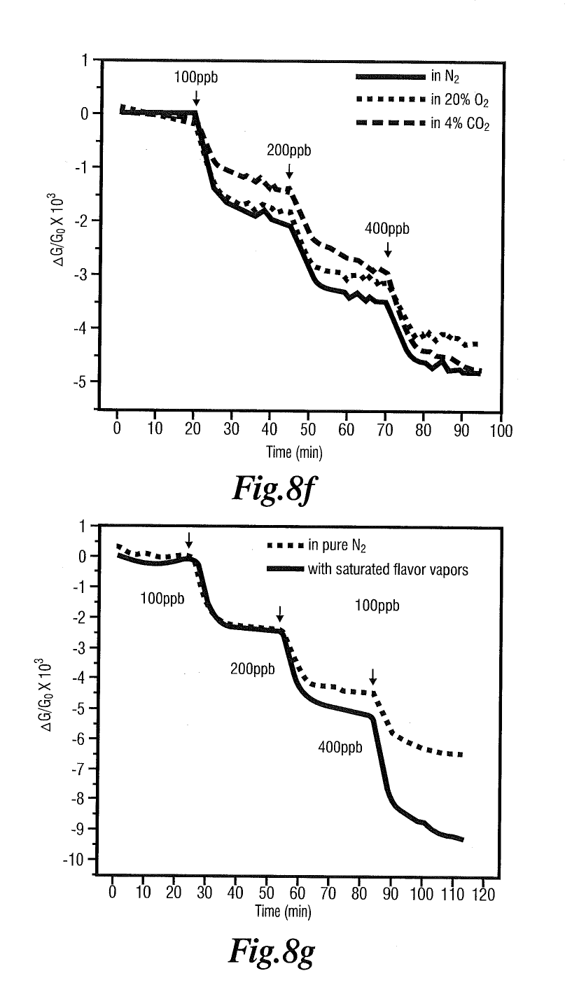

Gold nanowire morphology was found to be very important for hydrogen sulfide detection. Compared to SWNT functionalized with gold nanoparticles by electrodeposition (gold nanoparticle-SWNT), gold nanowire-SWNT showed better response (which is a decrease in conductivity) when exposed to hydrogen sulfide at the concentration range from 10 ppb to 40 ppm (diluted in nitrogen). FIG. 8 illustrates the method for preparation of gold-nanowire functionalized nanotubes and the difference in response between gold-nanowire functionalized nanotubes and gold-nanoparticles. The detection limit appeared to be lower than 10 ppb in nitrogen environment at room temperature, which meets the sensitivity requirement for the application of breath odor detection. FIG. 8a illustrates the preparation of gold nanowire functionalized nanotubes. The gold nanoparticles form nanowires by nucleation and assembly around the SWNTs. FIG. 8b illustrates a TEM image of gold nanowire functionalized nanotubes. FIG. 8c is a schematic representation of hydrogen sulfide sensing using gold nanowire functionalized nanotubes. FIG. 8d illustrates the relative conductance (ΔG/Gₒ) response of gold nanowire functionalized nanotubes upon exposure to increasing hydrogen sulfide pulses in concentrations from 10 ppb to 40 ppm. Arrows in FIG. 8d correspond to the point when hydrogen sulfide gas was introduced into the chamber.

FIG. 8e compares gold nanowire functionalized nanotubes of a preferred embodiment of the present invention with gold nanoparticle nanotubes at concentrations of 10 ppb to 100 ppb. The gold nanowire functionalized nanotubes showed a better detection limit than the gold nanoparticle nanotubes as seen by the greater decrease in relative conductance for the gold nanowire functionalized nanotubes at all hydrogen sulfide concentrations tested.

The cross sensitivity of gold nanowire functionalized nanotubes of a preferred embodiment of the present invention to other major components usually found in breath (oxygen, carbon dioxide, water, ethanol and artificial flavor compounds) were also tested as shown in FIGS. 8f and 8g. FIG. 8f illustrates the relative conductance response of gold nanowire functionalized nanotubes upon exposure to three hydrogen sulfide pulses in an atmosphere of nitrogen in comparison with pulses of hydrogen sulfide in atmospheres of 20% oxygen in nitrogen and 4% carbon dioxide in nitrogen. No obvious cross sensitivity to oxygen or carbon dioxide was observed. FIG. 8g illustrates the relative conductance response of gold nanowire functionalized nanotubes upon exposure to three hydrogen sulfide pulses in an atmosphere of nitrogen in comparison with pulses of hydrogen sulfide in the presence of saturated flavor vapors in nitrogen. The flavor vapors were a mixture of ten unknown flavor components received from the Colgate Palmolive Company. No obvious cross sensitivity to the saturated flavor vapors was observed.

### Example 2

### “X”=Polyaniline (PAni) Coatings With Additional Metal Salt Doping

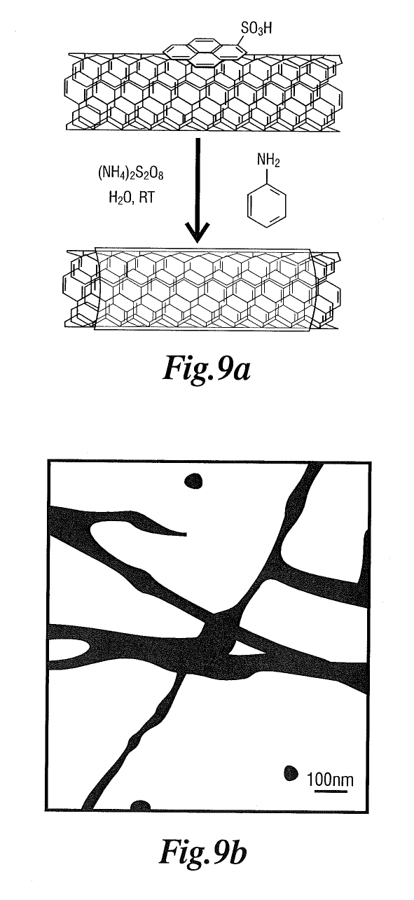

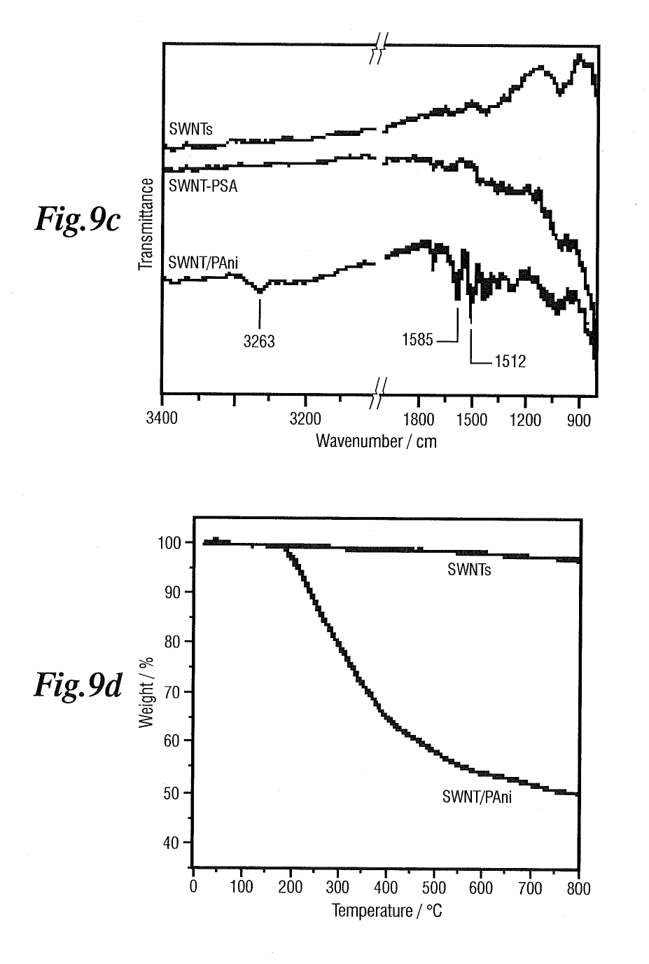

SWNT/PAni composite of a preferred embodiment of the present invention was synthesized by aniline polymerization in SWNTs suspension. SWNTs were functionalized with 1-pyrenesulfonic acid (PSA) prior to the polymerization as illustrated in FIG. 9a.

TEM and atomic force microscopy (AFM) of SWNT/PAni composite revealed the SWNTs had a uniform PAni coating (FIG. 9b). The PAni coating was further confirmed by Fourier transform infrared (FTIR) spectroscopy (FIG. 9c), where SWNT/PAni composite showed typical absorption peaks at 1512, 1585 and 3263 cm⁻¹. These bands were associated with stretching of benzenoid and quinoid rings as well as N—H stretching of benzenoid amine groups of PAni, respectively. Thermogravimetric analysis (FIG. 9d) confirmed that the ratio of PAni to SWNT was about 1:1 (wt), as the SWNT/PAni composite showed 50% mass loss from 200 to 800° C. This loss was attributed to the decomposition of PAni, as SWNTs remained stable in this temperature range.

Cupric chloride was incorporated into SWNT/PAni of a preferred embodiment of the present invention and the resulting composite was tested with hydrogen sulfide. As shown in FIG. 10, SWNT/PAni has a very weak response to 500 ppb hydrogen sulfide (introduced at arrow), which was comparable to bare SWNTs (FIG. 9d). When the copper (II) salt was incorporated, SWNT/PAni showed a significant increase in the hydrogen sulfide sensitivity, similar to what was reported for PAni/CuCl₂.

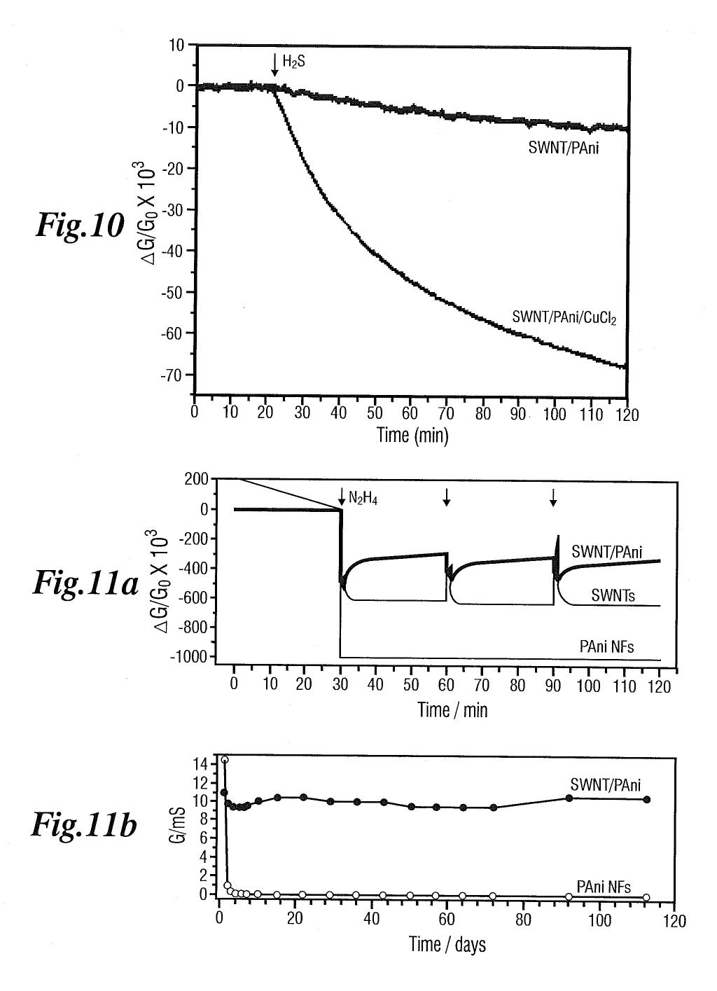

In comparison to bare SWNTs or PAni-nanofibers, SWNT/PAni composite of a preferred embodiment of the present invention demonstrated superior chemical stability. SWNT/PAni composite demonstrated a significant decrease in conductance upon exposure to hydrazine, similar to PAni-nanofibers (FIG. 11a). FIG. 11b depicts the monitoring of the baseline conductance of the SWNT/PAni composite and the PAni-nanofibers during continuous hydrazine tests over the course of four months. While PAni-nanofibers lost their conductivity after their first exposure and demonstrated no visible recovery, SWNT/PAni composite responded reversibly for four months of testing.

Materials. Pristine single-walled carbon nanotubes (SWNTs) were obtained from Carbon Solutions Inc. 1-pyrenesulfonic acid hydrate (1-PSA, C₁₆H₁₀O₃S. xH₂O), aniline, ammonium persulfate and all organic solvents were purchased from Sigma Aldrich and used as received. Lead ceramic sidebraze (CERDIP) packages (cavity 0.310×0.310) were procured from Global Chip Materials, LLC.

### Preparation of Different Graphitic Templates:

PSA functionalized SWNTs (SWNT-PSA). SWNTs powder (5.0 mg) was sonicated in distilled water (10 mL) for several minutes to obtain a temporary SWNTs suspension. Aqueous solution of 1-pyrenesulfonic acid (10 mL, 0.5 mg/mL) was then added into this SWNTs suspension following by bath sonication for 30 minutes. The resulting SWNT-PSA complex was separated out by centrifugation and washed with distilled water three times to afford the final product which was resuspended in 20 mL of distilled water.

Synthesis of SWNT/PAni: Aniline (0.1 mmol) was added into an aqueous (10 mL) suspension of PSA-functionalized SWNTs. An aqueous solution of ammonium persulfate (0.1 mmol) was then poured into the aniline and SWNT-PSA solution followed by vigorous stirring for 0.5-3 hours. The final product was isolated by filtration, washed with distilled water and ethanol several times, and resuspended in 20 mL of tetrahydrofuran.

Graphene oxide (GO) and chemically converted graphene (CCG). Graphite oxide was prepared on graphite flakes that had undergone a preoxidation step by a modified Hummer's method, Chem. Mater. 11, 771-78 (1999). Graphene oxide (˜0.125 wt %) was formed from the graphite oxide diluted 1:4 with double distilled water, exfoliated for 45 minutes by ultrasonication, and centrifuged for 30 minutes at 3400 revolutions per minute (r.p.m.) to remove unexfoliated graphite oxide. Chemically converted graphene (CCG) was prepared from the graphene oxide wherein a 75 gram dispersion of graphene oxide in water was adjusted to pH 9 with sodium carbonate, with a partial reduction through the addition of 600 mg of sodium borohydride. After heating at 80° C. for 1 hour under constant stirring, the material underwent four sequential washing/centrifugation cycles, and the partially reduced graphene oxide was dispersed in water via mild sonication for a final mass of 75 grams. Finally, the dispersion of partially reduced graphene oxide was reduced by four grams of 50 wt % hydrazine hydrate added to the dispersion, which was subsequently refluxed at 100° C. for 24 hours under constant stirring. After refluxing, the CCG was subjected to four sequential washing/centrifugation cycles.

PSA functionalized CCG (CCG-PSA). CCG-PSA was synthesized using the same method for the synthesis of SWNT-PSA.

### Self-Assembly and Nanowelding of Gold Nanoparticles:

Using SWNT-PSA template (Synthesis of gold nanowires functionalized SWNTs). Chloroauric acid (0.5 mg) was dissolved in an aqueous suspension of PSA-functionalized SWNTs (19 mL, 0.025 mg/mL). An aqueous solution of sodium citrate (1 wt %, 1 mL) was then added into chloroauric acid and SWNT-PSA solution with vigorous stirring. The mixture solution was heated to about 100° C. for 30-120 min to yield a purple suspension of SWNTs. The final product was isolated by centrifugation and washed with distilled water for several times, and then resuspended in 20 mL of distilled water. A control experiment was run under similar conditions without the presence of carbon nanotubes in solution.

Using other graphitic template: Self-assembly and nanowelding process on other graphitic templates (including CCG-PSA) was carried out with a same procedure to that of SWNT-PSA template.

Gold nanoparticles functionalized SWNTs by electrochemical deposition. Chloroauric acid solution (1 mM) was prepared with a supporting electrolyte of HCl (0.1 M). This solution was used to electrochemically deposit gold nanoparticles onto the pre-made SWNT network device (by drop casting 70 microliters DMF solution of SWNTs onto a Si chip with interdigitated gold electrodes which connected to ceramic dual in-line packages with gold wires and allowed to dry in vacuum oven). To carry out the electrochemical deposition a pair of stainless steel tweezers was used to bridge the source and drain bulk contacts of one particular device. This was connected to a CH Instruments Model 600 electrochemical analyzer and used as the working electrode. Connecting the device in this manner allowed for the entire SWNT network between the Source-Drain electrodes to function as a working electrode. A small drop (˜100 microliters) of metal solution was placed on top of the device and an Ag/AgCl reference and a Pt wire counter electrode were brought into contact with the surface of the solution. This configuration allowed the drop to act as a miniaturized 3-electrode electrochemical cell; the deposition was conducted under ambient conditions. After electrochemical decoration the devices were dipped in sequential baths of pure distilled water and dried in a vacuum oven at 75° C. overnight.

Platinum nanoparticles/nanowires functionalized SWNTs. Chloroplatinic acid (1 mL, 1-10 mM) was added into SWNT-PSA solution (18 mL) To the resulting solution, freshly made sodium borohydride (1 mL, 1 mg/mL) was then added at room temperature. The mixture was stirred at room temperature for 30 min. The final product was isolated by centrifugation and washed with distilled water for several times, and then resuspended into 20 mL of DI water.

General Characterizations. TEM of synthesized samples were performed operating at an acceleration voltage of 80 keV. High resolution TEM (HRTEM) images were obtained at an accelerating voltage of 200 keV. Scanning electron microscopy (SEM) was performed with a microscope equipped with an energy dispersive x-ray spectroscopy (EDS) accessory.

Spectroscopic Measurement. UV-vis-NIR absorption spectra studies were carried out on a UV-vis-NIR spectrophotometer Thin films used in the spectroscopic study were created by spraying a DMF suspension of the desired materials by airbrush onto a quartz plate (1″×1″) heated up to 180° C. A Teflon chamber was used to allow the gas flow exposure during spectroscopic measurements.

Sensor Device Fabrication. Si chips with 300 nm thermal oxide layer and interdigitated gold electrodes were purchased from MEMS and Nanotechnology Exchange The devices were fabricated by drop-casting aqueous suspension (10 microliters) of the gold-nanoparticle-SWNTs or gold-nanowire-SWNTs or DMF suspensions (70 microliters) of SWNTs onto the Si chips which were connected to ceramic dual in-line packages with gold wires and allowed to dry in ambient.

Gas Sensing Measurements. Gas sensing measurements were carried out on a custom-made system as in Kauffman, et al, Nano Lett. 7, 1863-68 (2007) incorporated herein. A Teflon chamber was used to control the gas environment during the sensing test. Different concentrations of analyte gases were generated by mixing certified gases (100 ppm hydrogen sulfide in nitrogen or 1 ppm dihyrogen sulfide in nitrogen) with dry nitrogen and were passed through the gas chamber containing the sensor device.

Breath Detection Measurements. Breath detection tests were done as above. The conductance of the sensor device was measured in ambient environment for the baseline, and for the breath test, a person's breathe was directed towards the device (duration 6-8 sec).

Features and advantages of single-wall carbon nanotube-based chemical sensors of preferred embodiments of the present invention include, but are not limited to the following. The sensors are small, allowing portable detectors and allowing the sensors to be incorporated into sensor arrays. By measuring electrical conductivity (resistivity) the device is a simple circuit. The sensor may be operated at room temperature, requiring no additional heating element. The sensors have low power consumption. The sensors are solid state, requiring no added electrolyte. The sensors should be low cost, allowing disposable products. For specific applications, there is no cross-sensitivity. The sensors have a low detection limit.

In the foregoing Detailed Description, various features are grouped together in a single embodiment to streamline the disclosure. This method of disclosure is not to be interpreted as reflecting an intention that the claimed embodiments of the invention require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

We claim:

1. A method for preparing a sulfide sensitive carbon allotrope, the steps comprising:
first functionalizing a carbon allotrope having sp² bonded carbon atoms with a polynuclear aromatic hydrocarbon-sulfonic acid, wherein a first-functionalized carbon allotrope is formed; and
second functionalizing the first-functionalized carbon allotrope with a metal to give the sulfide sensitive carbon allotrope.

2. The method of claim 1, wherein the polynuclear aromatic hydrocarbon-sulfonic acid is 1-pyrene sulfonic acid.

3. The method of claim 1, wherein the metal comprises a noble metal; and wherein the second functionalizing comprises a deposition of the noble metal and nanowelding the noble metal into noble metal nanowire from the metal.

4. The method of claim 3, wherein the noble metal comprises gold.

5. The method of claim 3, wherein nanowelding comprises heating.

6. The method of claim 1, wherein the second functionalizing comprises an aniline polymerization and doping with a cupric (II) salt.

7. The method of claim 6, wherein the cupric (II) salt comprises cupric chloride.

8. The method of claim 1, wherein the carbon allotrope comprises a carbon nanotube.

9. The method of claim 1, wherein the carbon allotrope comprises graphene.

10. A method of formation of a sulfide sensor, the method comprising the steps of:
first functionalizing a carbon allotrope having sp² bonded carbon atoms with a polynuclear aromatic hydrocarbon-sulfonic acid, wherein the first-functionalized carbon allotrope are formed;
second functionalizing the first-functionalized carbon allotrope with a metal, whereby a sulfide sensitive carbon allotrope is formed; and
depositing the sulfide sensitive carbon allotrope onto an inert surface.

11. The method of claim 10, wherein the inert surface comprises silicon dioxide.

12. The method of claim 10, wherein the inert surface comprises silicon dioxide containing at least one electrode.

13. A composite comprising:
a carbon allotrope having a first and second opposing surfaces and comprising sp² bonded carbon atoms;
a polynuclear sulfonic acid disposed on the first surface;
and a sulfide reactive material substantially disposed on the polynuclear sulfonic acid.

14. The composite of claim 13, wherein the sulfide reactive material comprises a noble metal nanowire.

15. The composite of claim 13, wherein the sulfide reactive material comprises a gold nanowire.

16. The composite of claim 13, wherein the sulfide reactive material comprises polyaniline doped with a cupric salt.

17. The composite of claim 13, wherein the sulfide reactive material comprises polyaniline doped with cupric chloride.

18. The composite of claim 13, wherein the carbon allotrope comprises a carbon nanotube.

19. The composite of claim 13, wherein the carbon allotrope comprises graphene.

20. The composite of claim 13, wherein the polynuclear aromatic hydrocarbon-sulfonic acid is 1-pyrene sulfonic acid.

21. A chemical sensor for sulfides, comprising.
a carbon allotrope comprising sp² bonded carbon atoms;
the carbon allotrope disposed on an inert surface;
a polynuclear sulfonic acid disposed on the carbon allotrope;
and a sulfide reactive material substantially disposed on the polynuclear sulfonic acid.

22. The chemical sensor of claim 20, wherein the inert surface comprises silicon dioxide.

23. The chemical sensor of claim 20, wherein the inert surface comprises silicon dioxide containing at least one electrode.

24. A method of use of the chemical sensor, comprising the steps of:
exposing a chemical sensor comprised of a carbon allotrope comprising sp² bonded carbon atoms, the carbon allotrope disposed on an inert surface, a polynuclear sulfonic acid disposed on the carbon allotrope, and a sulfide reactive material substantially disposed on the polynuclear sulfonic acid; and
measuring the response of the sensor to the sulfide compound.

25. The method of claim 24, wherein measuring the response comprises an evaluation of a spectrophotochemical change of the chemical sensor.

26. The method of claim 24, wherein measuring the response comprises evaluating a change in conductance between at least two electrodes on the chemical sensor.
